(19) United States
(12) Patent Application Publication
(10) Pub. No.: US 2008/0248117 A1
(43) Pub. Date: Oct. 9, 2008

(54) Production of Solid Solutions Based on Poorly-Soluble Active Substances by a Short-Term Heating and Rapid Drying

(75) Inventors: Karl Kolter, Limburgerhof (DE); Michael Schoenherr, Frankenthal (DE); Hermann Ascherl, Dirmstein (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/916,074

(22) PCT Filed: May 31, 2006
(86) PCT No.: PCT/EP2006/062788

§ 371 Date: Nov. 30, 2007

(30) Foreign Application Priority Data
Jun. 9, 2005 (DE) 10 2005 026 755.6

(51) Int. Cl. A61K 9/00

(52) U.S. Cl. 424/486; 424/484

(57) ABSTRACT

A process for producing solid solutions in powder or granule form of slightly soluble substances in which the slightly soluble substance is in the form of a molecular dispersion in an excipient matrix, by atomizing a solution of the active ingredient and of the matrix excipients, which comprises heating an aqueous suspension of the slightly soluble substance in the presence of the matrix excipients to temperatures above the boiling point under atmospheric pressure, and dissolving the slightly soluble substance, and subsequently converting the solution of the slightly soluble substance and of the matrix excipients by atomizing and drying into a solid form, where the temperature of the spray solution before feeding into the atomizing apparatus is 90° C. to 350° C.

The present invention relates to a process for producing solid solutions of slightly soluble active ingredients, to products in powder form which are obtained by such a process, and to the use thereof for dosage forms.

Active ingredients which are slightly soluble in aqueous media are extremely difficult to formulate because it is scarcely possible to develop bioavailable dosage forms. One of the most promising approaches is to form solid solutions in which the active ingredient is incorporated in the form of a molecular dispersion. This form very often leads to distinctly higher bioavailabilities because, after the polymer has dissolved, the active ingredient is available in dissolved form to the body or the plant.

The term solid solution is often incorrectly used in the literature, because incorporations of solid crystalline substances are also referred to as solid solution. Strictly speaking, however, these are solid dispersions. In this document, solid solution means a genuine molecular dispersion.

Various substances said to be suitable for producing solid solutions have already been described, in particular also polymers such as polyvinylpyrrolidones, cellulose esters, sugars, sugar alcohols, starches and natural polysaccharides.

The production of such solid solutions is still a rather complicated process.

The following methods are available:
1. melting of active ingredient and polymer at high temperature and extrusion. This process has the disadvantage that high temperatures act on the active ingredient for some minutes, and in addition large shaped articles result and must be laboriously comminuted by grinding in order to be tabletable. Moreover, the shaped articles generally have no porosity, so that compressibility is very poor. The tablets frequently show low resistance to crushing and high friability. Only products with a certain minimum porosity can be compressed easily. An additional factor is that, besides the thermal stress, shear forces also act during melt extrusion and may lead to decomposition of the active ingredients.
2. dissolving of active ingredient and polymer in an organic solvent which dissolves both, and evaporation of the solvent or spray drying. This process has the disadvantage per se that it is necessary to employ organic solvents on a large scale, which are dangerous to the environment and explosive and whose use causes considerable costs. In addition, it is often difficult to find a solvent which dissolves both the hydrophilic polymer and the lipophilic medicinal substance.
3. dispersing of active ingredient in an aqueous polymer solution, for example by wet grinding and spray drying. If in this case the active ingredient is not soluble in water there is formation not of solid solutions but only of solid dispersions which are far from having the properties like molecular solutions, especially not in relation to bioavailability.

U.S. Pat. No. 5,876,760 describes spray-dried powders composed of pranlukast, saccharides, if appropriate water-soluble polymers and surfactants. The active ingredient is suspended in an aqueous solution of the excipients, and the latter is spray dried under conventional conditions. The active ingredient is present in the final product in crystalline form.

U.S. Pat. No. 6,197,781 describes a rapamycin-containing formulation where the active ingredient is dissolved in a solvent and is spray dried together with a carrier which may consist of polyoxyethylene-polyoxypropylene block copolymer, polyvinylpyrrolidone, microcrystalline cellulose and a water-soluble saccharose, at temperatures of 20-80° C. Large amounts of organic solvents are used in this case, and the temperatures of the solution before spray drying are distinctly below 100° C.

WO 99/56751 describes amorphous paroxetine formulations which are produced by mixing paroxetine salts, preferably the hydrochloride, with water and a polymer and subsequently drying at 25-100° C., preferably at 60° C. The paroxetine salts are soluble in water even at low temperatures, so that production of a solid solution does not represent a special problem. WO 01/30349 likewise relates to the processing of amorphous paroxetine salts in polyvinylpyrrolidone and an additional acid. Production takes place at temperatures of 15-40° C.

WO 03/000294 relates to solid dispersions of poorly water-soluble medicinal substances in a matrix with a solubility-enhancing polymer. Production by using organic solvents or by melt processing is described for example.

DE 19951617 is concerned with pharmaceutical dosage forms with an active ingredient in two different physical forms. In this case, the active ingredient is partly in particulate form and in dissolved form. Production can take place in a conventional way employing organic solvents. Production of the solid solution preferably takes place by melt extrusion.

US 2001/0007678 and US 2003/0082239 describe solid dispersions of itraconazole with water-soluble polymers produced by melt extrusion and subsequent grinding. These preparations have the previously known disadvantages such as, for example, poor tabletability.

The solid dispersions described in US 2002/0009494 are produced by dissolving a slightly soluble medicinal substance together with hydroxypropylmethylcellulose acetate succinate in an organic solvent, and spray drying. Unusually large amounts of solvent are required to produce one part of preparation.

US 2002/0031547 discloses the dissolving of medicinal substances with a gel-forming water-soluble polymer in an organic solvent and drying. This preparation is then mixed with a salt of alkali and a weak or strong acid, and tableted. The gel-forming polymers are cellulose ethers. Since gel-forming polymers slow down the disintegration and release of active ingredient from tablets, it is not surprising that further additives are necessary to increase the rate of disintegration.

WO 01/47492 describes solid dispersions of practically insoluble medicinal substances with polymers which have acidic functional groups. The medicinal substance is in the particulate form, and organic solvents, especially methylene chloride, are used for the production.

WO 2002051385 likewise describes the production of solid solutions using organic solvents. The polymers employed are cellulose derivatives and polyvinylpyrrolidone. Further excipients are wetting agents.

Further solid solutions using organic solvents are described in WO 2001068092, EP 1027886, EP 102787, EP 102788, US 2001/0053778, WO 03/000235, WO 2001068092, WO 2003000226, US 2001/0053791.

US 2003/0104068 and US 2003/082236 disclose incorporations of active ingredient particles which have a size of less than 2 μm. Because of the particulate state, a solid solution in the real sense is not involved.

DE 4329446 describes a process in which a melt emulsion of an active ingredient in water or mixtures of water with organic solvents is produced above the melting point of the active ingredient in the presence of a protective colloid, and this emulsion is spray dried. The result in this case is colloidal active ingredient particles and not solid solutions.

It was an object of the present invention to find a process which avoids organic solvents, does not involve great thermal stress for the active ingredients, directly affords a product with good tabletability and flowability, and is easy to carry out.

Accordingly, a process for producing solid solutions in powder form of slightly soluble substances in which the slightly soluble substance is in the form of a molecular dispersion in an excipient matrix, by atomizing a solution of the slightly soluble substance and of the matrix excipients, has been found and comprises heating an aqueous suspension of the slightly soluble substance in the presence of the matrix excipients under pressures of from 0.08 to 20 MPa to temperatures of >90° C. to 350° C., and dissolving the slightly soluble substance, and subsequently converting it by atomizing and drying into the form of a powder, where the temperature of the spray solution when fed into the atomizing apparatus is >90 to 350° C.

A solid solution designates according to the invention a state in which the active ingredient is in the form of a molecular dispersion in a matrix of excipients. In this state, no crystalline fractions of the active ingredient are detectable by X-ray diffractometry. Since the limit of detection for crystalline fractions in X-ray diffractometry is 3% by weight, the expression “no crystalline fractions” means that fewer than 3% by weight crystalline fractions are present. The state of molecular dispersion can be ascertained by means of the method of differential scanning calorimetry (DSC). In the case of a molecular dispersion, no melting peak is to be observed in the region of the melting point of the active ingredient. The limit of detection of this method is 1% by weight.

Slightly soluble substances mean in the context of the invention substances whose saturation solubility at room temperature (20° C.) is less than 1% by weight in at least one of the following media: water, 0.1 molar aqueous hydrochloric acid, aqueous phosphate buffer of pH 7.2, 0.9% by weight aqueous sodium chloride solution.

Suitable slightly soluble substances according to the invention are a large number of active ingredients and active substances, especially pharmaceutical or cosmetic active ingredients, active ingredients for dietary supplements or dietetic compositions or food additives.

Examples of slightly soluble substances in the context of the invention are:

piroxicam, clotrimazole, carbamazepine, 17-beta-estradiol, sulfathiazole, fenofibrate, benzocaine, lidocaine, dimethindene, biperiden, bisacodyl, clioquinol, droperidol, haloperidol, nifedipine, nitrendipine, tetracycline, phenytoin, glafenine, floctafenine, indometacin, ketoprofen, ibuprofen, dipyridamole, mefenaminic acid, amiodarone, felodipine, itraconazole, ketoconazole, danazole, furosemide, tolbutamide, ritonavir, lopinavir, naproxen, spironolactone, propafenone, progesterone, paclitaxel, docetaxel, theophylline, hydrocortisone, beta-carotene, vitamin A, tocopherol acetate, riboflavin, vitamin Q 10, vitamin D, vitamin K, disulfiram, nimodipine, chlorthiazide, chlorpropamide, dicoumarol, chloramphenicol, digoxin, lonidamine, pizotifen, atovaquone, amprenavir, bexarotene, calcitrol, clofazimine, doxercalciferol, dronabinol, durasteride, etoposide, loratadine, risperidone, saquinavir, sirolimus, valproic acid, amphotericin, alprostadil, carmustine, chlordiazepoxide, fenoldopam, melphalan, methocarbamil, oxytetracycline, docetaxel, fulvestrant, propofol, voriconazole, ziprasidone, leuprolide acetate, viadur, valrubicin, tramadol, celecoxib, etodolac, refocoxib, oxaprozin, leflunomide, diclofenac, nabumetone, ibuprofen, flurbiprofen, tetrahydrocannabinol, capsaicin, ketorolac, albendazole, ivermectin, amiodarone, zileuton, zafirlukast, albuterol, montelukast, azithromycin, ciprofloxacin, clarithromycin, dirithromycin, rifabutin, rifapentin, trovafloxacin, baclofen, ritanovir, saquinavir, nelfinavir, efavirenz, dicoumarol, tirofibran, cilostazol, ticlidopine, clopidrogel, oprevelkin, paroxetine, sertraline, venlafaxine, bupropion, clomipramine, miglitol, repaglinide, glymepride, pioglitazone, rosiglitazone, troglitazone, glyburide, glipizide, glibenclamide, fosphenytion, tiagabine, topiramat, lamotrigin, vigabatrin, amphotericin B, butenafine, terbinafine, itraconazole, flucanazole, miconazole, ketoconazole, metronidazole, griseofulvin, nitrofurantoin, lisinopril, benezepril, nifedipine, nilsolidipine, telmisartan, irbesartan, eposartan, valsartan, candesartan, minoxidil, terazosin, halofantrine, mefloquine, dihydroergotamine, ergotamine, frovatriptan, pizotifen, sumatriptan, zolmitriptan, naratiptan, rizatriptan, aminogluthemide, busulphan, cyclosporin, mitoxantrone, irinotecan, etoposide, teniposide, paclitaxel, tacrolimus, sirolimius, tamoxifen, camptothecan, topotecan, nilutanide, bicalutanide, toremifen, atovaquone, metronidazole, furazolidone, paricalcitol, benzonatate, midazolam, zolpidem, gabapentine, zopiclone, digoxin, beclomethasone, budesonide, betamethasone, prednisolone, cisapride, cimetidine, loperamide, famotidine, lanosprazole, rabeprazole, nizatidine, omeprazole, citrizine, cinnarizine, dexchiopheniramine, loratadine, clemastine, fexofenadine, chlorpheniramine, acutretin, tazarotene, calciprotiene, calcitriol, targretin, ergocalciferol, cholecalciferol, isotretinoin, tretinoin, calcifediol, fenofibrate, probucol, gemfibrozil, cerivistatin, pravastatin, simvastatin, fluvastatin, atorvastatin, tizanidine, dantrolene, isosorbide dinatrate, dihydrotachysterol, essential fatty acids, codeine, fentanyl, methadone, nalbuphine, pentazocine, clomiphene, danazole, dihydroepiandrosterone, medroxyprogesterone, progesterone, rimexolone, megesterol acetate, estradiol, finasteride, mefepristone, L-thryroxine, tamsulosine, methoxsalen, tacrine, donepezil, raloxifene, vertoporfin, sibutramine, pyridostigmine, and their isomers, derivatives, salts or mixtures.

The solid solutions produced by the process of the invention may have the following quantitative composition:
(i) 1 to 50% by weight of at least one active ingredient,
(ii) 10 to 99% by weight of at least one water-soluble matrix excipient,
(iii) 0 to 30% by weight of one or more surfactants,
(iv) 0 to 30% by weight of cosolubilizers,
(v) 0 to 50% by weight of further excipients,

where the amounts of components (i) to (v) add up to 100% by weight.

Suitable matrix-constructing excipients are in principle all substances able to form solid solutions with active ingredients.

Suitable examples are water-soluble polymers from the following structural classes:
polyvinylpyrrolidones, vinylpyrrolidone-vinyl acetate copolymers,
polyvinylcaprolactams, polyvinylformamide, polyvinylacetamide, polyacrylates,
polymethacrylates, polyacrylamides, polyethyleneimines, polyvinylamines hydroxyalkylcelluloses, alkylhydroxyalkylcelluloses, carboxyalkylcelluloses,
alkylhydroxyalkylcellulose acetate succinates, alkylhydroxyalkyicellulose acetate phthalates, alkylhydroxyalkylcellulose phthalates, cellulose acetate phthalates starches, hydroxyalkylstarches, carboxyalkylstarches, modified starch,
octenyisuccinate-starches,
dextrans,
polyoxyethylene-polyoxypropylene block copolymers,
polyethylene oxides, polypropylene oxides,
polyamino acids

It is, however, also possible to employ low molecular weight carriers:
sugars such as sucrose, glucose, maltose, xylose, fructose, ribose, arabinose,
galactose, trehalose
sugar alcohols such as sorbitol, mannitol, xylitol, erythritol, patatinol, maltitol, lactitol
urea
nicotinamide
amino acids
cyclodextrins

Preferred substances are those having amide structures because they are able to dissolve high concentrations of active ingredients.

The polymeric matrix excipients preferably used are polyvinylpyrrolidone, copolymers of N-vinylpyrrolidone and vinyl acetate. It is also preferred to use alkyl methacrylates or alkyl acrylates.

The Fikentscher K values of the polymers in a 1% by weight aqueous solution may be from 5 to 120, preferably 10 to 95.

Urea is a particularly suitable low molecular weight matrix excipient.

The matrix excipients (ii) are preferably employed in amounts of from 30 to 90% by weight.

It is possible additionally to employ solubilizers for further improving the solubility. Suitable solubilizers are surfactants, which ordinarily have a HLB above 10 (HLB: hydrophilic lipophilic balance). Such solubilizers are described in: Fiedler, Lexikon der Hilfsstoffe, Editio Cantor Verlag Aulendorf, 5^{th} edition, page 117-121

The following have proved particularly suitable:
alkali metal or ammonium salts of fatty acids, alkali metal or ammonium salts of sulfonated or sulfated fatty acids, polyoxyethylene fatty acid esters, polyoxyethylene fatty alcohols, polyoxyethylene glycerol fatty acid esters,
polyoxyethylene glycerol fatty alcohols, polyoxyethylenesorbitan fatty acid esters, ethoxylated castor oil, ethoxylated hydrogenated castor oil, ethoxylated 12-hydroxystearic acid, poloxamers or mixtures thereof.

Such surfactants (iii) are preferably employed in amounts of from 1 to 20% by weight.

It has also proved advantageous in particular cases to use cosolubilizers (iv) with a HLB below 10, because the formation and the stability of the solid solution is promoted thereby. These substances are likewise described in: Fiedler, Lexikon der Hilfsstoffe, Editio Cantor Verlag Aulendorf, 5^{th} edition, page 115-117

The following substances can be employed for example:
polyoxyethylene fatty acid esters, polyoxyethylene fatty alcohols, polyoxyethylene glycerol fatty acid esters, polyoxyethylene glycerol fatty alcohols,
glycerol fatty acid esters, glycerol fatty alcohols, sorbitan fatty acid esters

It may also be advisable to employ organic solvents in amounts of up to 10% by weight as additional solubilizers. Suitable organic solvents are ethanol, isopropanol or acetone. However, it is preferred to dispense with the use of organic solvents.

Some of the active ingredients, solubilizers and cosolubilizers have a considerable plasticizing effect, i.e. they reduce the glass transition temperature of the polymer distinctly, thus occasionally making the spray drying difficult. In these cases, it has proved very advantageous to use an adsorbent. This adsorbent absorbs the liquid or semisolid active ingredient polymer solution and thus produces a solid preparation which can be used satisfactorily. Examples of adsorbents which can be employed are the following substances: silica, hydrophobic silica, alkali metal or alkaline earth metal silicates, alkaline earth metal/aluminum silicates, crosslinked polyvinylpyrrolidone, cellulose, starch, crosslinked sodium carboxymethylstarch, crosslinked sodium carboxymethylcellulose.

The adsorbent is ordinarily suspended in the spray solution before the heating step, and is dried therewith. However, a portion may also be blown in powder form into the spray tower.

It is additionally possible, in order to achieve specific characteristics, to use further excipients (v).
plasticizers
antioxidants
preservatives
colors
flavorings and odorants
fillers,
antistick agents
disintegration-promoting excipients (disintegrants)
release-slowing agents

Excipients of these types are preferably present in amounts of from 0.1 to 20% by weight.

According to the invention, aqueous solutions comprising the active ingredient and the matrix excipients and, if appropriate, the further components (iii) to (v) are initially prepared by heating. Water is preferably used as the only solvent.

The following methods are available in principle for preparing the solutions:
Method A: an aqueous suspension which comprises the active ingredient in suspended form and the matrix excipients in dissolved form, and if appropriate the further components, is prepared. This can be done either by initially dissolving the matrix excipients in water and suspending the active ingredient in this solution, or adding the matrix excipients to an aqueous suspension of the active ingredient. The suspension obtained in this way is then heated in a suitable apparatus until the active ingredient has dissolved.

Method B: an aqueous suspension of the active ingredient which comprises the matrix excipients in dissolved form is prepared as described for method A, and this suspension is heated by mixing with a hot stream of water or a stream of steam until the active ingredient has dissolved.

Method C: in a slight modification of method B, it is also possible for matrix excipients, as long as they are thermally stable, to be dissolved in a hot stream of water and be mixed with a suspension of the active ingredient in water.

The following applies, irrespective of the method chosen:

A small particle size is advantageous for dispersing the active ingredient in water or the aqueous polymer solution, because it firstly facilitates the dispersing and secondly the dissolving process at elevated temperature is faster. If a coarse active ingredient is placed, this can also be reduced in size or ground in the polymer solution before the suspension is heated. It is possible to use for the size reduction for example high pressure homogenizers, rotor-stator equipment, ball mills or colloid mills. However, it is also possible in principle for the active ingredient as described to be put into water first, and only then for the polymer to be added.

The heating of the aqueous suspension takes place continuously in a suitable apparatus.

The heating can take place for example in any suitable heat exchanger, where apparatuses referred to as heat exchangers are generally those in which heat is transported by a heat-transfer agent to another medium in order to achieve heating.

In indirect heat exchange, the heat-transfer medium and the medium to be heated are separated by heat-exchange surfaces. Suitable as heat-transfer medium are hot oil, hot vapor or superheated water or else generally hot gases or hot liquids. The heat-transfer medium can be passed counter-currently to the aqueous suspension to be heated. A further possibility is also to pass the medium to be heated continuously through a static heat-transfer medium.

In direct heat exchange, as takes place according to the invention in methods B) or C), the two media are in contact. Suitable direct heat-exchange agents are therefore superheated water or steam as heat-transfer agents.

The heating of the active ingredient suspension can generally take place using all processes which make a very rapid heating rate possible. Thus, electrical, inductive or microwave heatings are also possible.

In order to dissolve the active ingredient in water, the aqueous suspension is heated to temperatures which are above the boiling point of the mixture under atmospheric pressure. Temperatures which can be chosen in this connection are from >90° C. to 350° C., preferably 110 to 300° C., particularly preferably 120 to 250° C.

In order to avoid thermal stress on the starting materials, irrespective of which of the described methods is used, the residence times on heating at >90° C. are kept in the region of seconds. The residence time of the active ingredient-containing medium in the apparatus employed for the heating is preferably less than 180 seconds, particularly preferably less than 60 seconds, very particularly preferably less than 15 seconds. In order to achieve complete dissolving of the active ingredient, generally a minimum residence time of 0.5 seconds is chosen.

The solids content of the solutions is normally from 1 to 70% by weight, preferably 3 to 60% by weight, particularly preferably 5 to 40% by weight.

The hot and pressurized aqueous solution of active ingredient, matrix excipients and if appropriate further components is passed after passage through the apparatus directly into an atomizing apparatus. The atomizing can take place through nozzles, in which case in principle single- or multiple-fluid nozzles are suitable, or by rotating disks. The atomization of the preparation in the drying tower preferably takes place through single-fluid nozzles under pressures of from 10 to 250 bar. However, multiple-fluid nozzles, in particular two-fluid nozzles, can also be employed, in which case the pressure of the atomizing gas can be from 0.15 to 10 MPa.

The tower inlet temperatures of the drying gas are between 50 and 200° C., preferably between 70 and 180° C. Suitable drying gases are air or inert gases such as nitrogen, argon or helium. The tower outlet temperatures are from 40 to 120° C. The drying gas can be passed co-currently or counter-currently to the liquid droplets in the drying tower, preferably co-currently.

Besides simple spray drying, it is also possible to carry out an agglomerating spray drying with internal and/or external fluidized bed (e.g. FSD Technologie from Niro), in which case the particles formed in the spray drying are agglomerated to larger structures.

It is generally possible to employ all drying techniques in which a solution is atomized, including fluidized bed spray granulation.

If the spray-dried particles show a certain tendency to adhere, dusting with a very fine-particle solid is appropriate. In this case, this fine-particle solid is introduced as dust into the spray tower and thus ensures that no adhesion or agglomeration takes place. Colloidal silica has proved very suitable here. However, it is also possible to employ other substances, for example hydrophobic silica, alkali metal or alkaline earth metal silicates, alkaline earth metal/aluminum silicates, crosslinked polyvinylpyrrolidone, cellulose, starch, crosslinked sodium carboxymethylstarch or crosslinked sodium carboxymethylcellulose.

In one embodiment for producing the solid solutions by method A according to the invention, the medicinal substance is accordingly dispersed in an aqueous solution of the polymer, and the suspension is heated in a suitable apparatus to temperatures above 90°, so that the active ingredient crystals dissolve. The heating of the active ingredient-containing polymer solution should take place as quickly as possible in order to minimize the thermal stress on the medicinal substance, For this purpose, the active ingredient-containing suspension is continuously passed through a suitable apparatus, with the residence times being, as described, preferably in the region of a few seconds. This heated and pressurized active ingredient solution is subsequently atomized and dried. The temperature of the spray solution shortly before the atomization, i.e. before introduction into the atomizing apparatus, is >90-350° C., preferably 110-300° C. and particularly preferably 120-250° C. The pressure of the spray solution is in this case from 0.08 to 20 MPa, preferably 1 to 15 MPa.

In a preferred embodiment of the invention, the active ingredient-containing polymer solution can be pumped through a thin pipeline which is located in a hot oil bath which has temperatures of 110-500° C., preferably 130-300° C. This makes rapid heat transfer possible. The temperature of the active ingredient-containing polymer solution is adjusted by varying the oil bath temperature and the flow rate. Immediately subsequent to passing through the pipeline, the hot, pressurized solution is atomized through a spray nozzle and dried with hot drying gas. The evaporation of the water results in abrupt cooling and drying of the spray droplets.

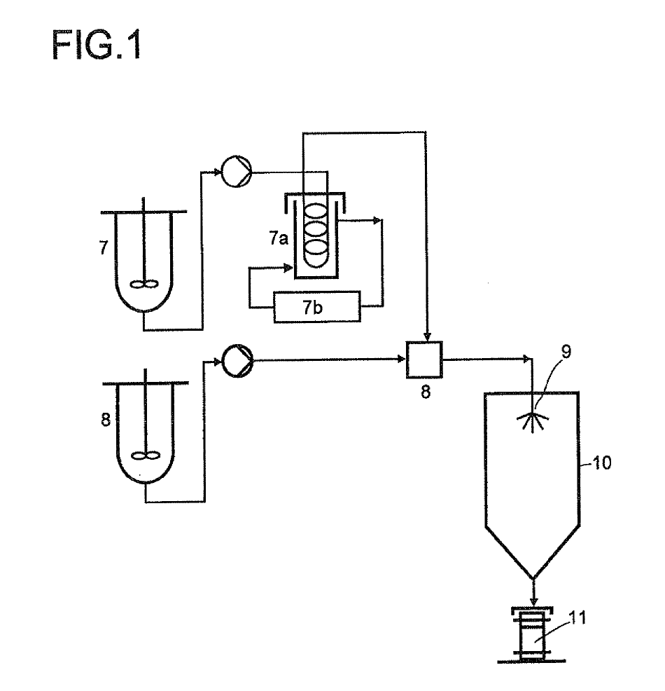

A procedure of this type is depicted diagrammatically for example in FIG. 1. In this case, the suspension of the active ingredient in the aqueous solution of the matrix excipients is prepared in a container 1 equipped with a stirrer, the suspension is then pumped continuously in a coiled pipe through a heat exchanger 2 which is equipped with a heater 2a to heat the heat-transfer medium, and the solution is subsequently atomized and dried through a nozzle 3 in a spray tower 4, and the resulting particulate solid solution 5 is collected.

In a further embodiment of the invention, it is possible to choose the procedure of method B described below. This procedure is particularly recommended when the thermal stress of the slightly soluble substance is to be further minimized. The slightly soluble substance is suspended in the polymer solution at room temperature or slightly elevated temperature at which the active ingredient is not decomposed. This suspension is fed into a mixing cell in which it is turbulently mixed with superheated water or steam. The temperature of the water or steam should be between 110-500° C., preferably 150-400° C., particularly preferably 180° C.-300° C. The high temperature of the water or steam and the turbulent mixing result in the suspension of the active ingredient in the polymer solution being heated in a very short time to temperatures above 100° C., and the active ingredient dissolving. Passing through the mixing cell is immediately followed by the atomization in a spray nozzle and the spray drying. The temperature of the solution to be sprayed is controlled through the temperatures of the two liquid streams and the ratio of mixing thereof. Higher temperatures of the water or steam stream and a larger ratio of water or steam stream to active ingredient/polymer suspension increase the temperature of the active ingredient solution to be sprayed. The residence time in the mixing cell depends on the flow rate of the two liquid streams and the geometry of the mixing cell. The suspension of the active ingredient in the polymer solution is ordinarily brought to the desired temperature within fractions of a second. The thermal stress on the active ingredient also depends on how quickly the spray drying follows the mixing. The distance between the mixing cell and spray nozzle ought therefore to be appropriately small. A minimum residence time is necessary to dissolve the active ingredient crystals and results from the active ingredient-specific rate of dissolution, the temperature of the solution or suspension and the particle size. The total residence time of the active ingredient at high temperatures can be adjusted through the flow rate, the geometry of the mixing cell and the length of the distance to the spray nozzle. The total residence time is ordinarily less than 30 seconds, preferably less than 15 seconds and particularly preferably less than 5 seconds.

If the rate of dissolution of the active ingredient is high, times of less than 1 second can also be attained.

The volumetric flows may be varied in the ratio from 9:1 to 1:9.

The geometry of the mixing cell may vary widely. From a simple T-piece to very refined cells mixing with high turbulence. The angle at which the streams are brought together may be between 5 and 180°. In a particular embodiment, one stream can be injected by means of an injector nozzle into the other stream.

Further excipients such as, for example, solubilizers are ordinarily introduced into the active ingredient-containing stream, but they can in principle also be fed in via the hot water phase.

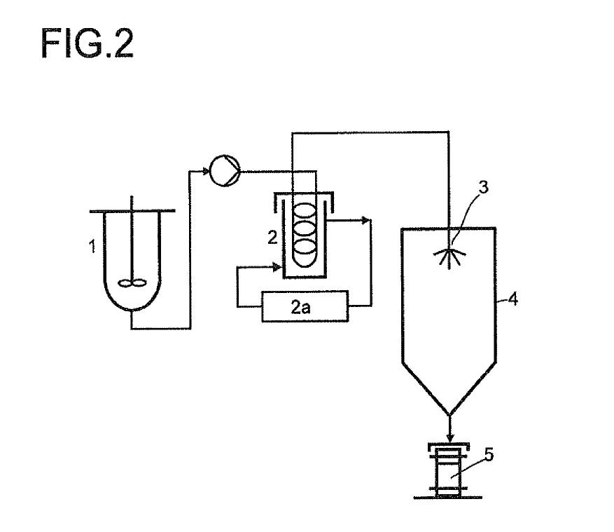

A procedure of this type is depicted diagrammatically in FIG. 2. In this case, an active ingredient suspension is prepared in a solution of the matrix excipients in a container 6 equipped with a stirrer and is pumped continuously into a mixing cell 8. Water is continuously pumped out of a container 7 through a heat exchanger 7a which is provided with a heater 7b, and is pumped as superheated water or steam likewise into the mixing cell 8. The heating and dissolving of the active ingredient takes place in the mixing cell 8 through continuous mixing of the two streams. The hot solution is then atomized through a nozzle 9 in a spray tower 10, and the particulate solid solution 11 is collected.

The powder produced by the process of the invention exhibits, owing to its porosity, very good tableting properties. Average particle sizes of from 25 to 500 μm are normally obtained.

The advantage of the preparations produced according to the invention is that high concentrations of active ingredient are in the form of a solid, molecular solution, so that the solid solution rapidly dissolves in aqueous medium and the active ingredient is kept for a long time in the supersaturated region in the aqueous medium. A large biological effect is achieved thereby.

The preparations of the invention exhibit excellent tabletability which is considerably better than on use of previously disclosed production processes. The resulting tablets have high resistance to crushing and very low friability. The preparations of the invention are ordinarily directly tabletable.

The active ingredient release can be controlled appropriately by adding a release-slowing agent. It is thus possible ideally to produce slow-release forms of slightly soluble active ingredients which exhibit very reproducible release.

### EXAMPLES

### Example 1

Solid solution of theophylline in povidone

10.0 kg of Kollidon 30 were dissolved in 40.0 kg of demineralized water. 5.0 kg of finely ground theophylline were suspended with vigorous stirring in this polymer solution. The brief heating took place by pumping the solution through a thin coiled pipeline with a diameter of 10 mm which was situated in an oil bath at a temperature of 155° C., during which the temperature of the solution rose to 150° C. The flow rate, which was set with a high-pressure pump, was 800-1000 ml/min. The pressure in the pipeline was 10.45 MPa. This hot solution was atomized through a single-fluid nozzle with a diameter of 0.6 mm under a pressure of 100 bar in a spray dryer. An outlet air temperature of 95° C. was set up with an inlet air temperature of 145° C. A dry, free-flowing powder was obtained.

### Example 2

Solid solution of carbamazepine in povidone

10.0 kg of Kollidon 30 were dissolved in 40.0 kg of demineralized water. 5.0 kg of finely ground carbamazepine were suspended with vigorous stirring in this polymer solution. The brief heating took place by pumping the solution through a thin coiled pipeline with a diameter of 10 mm which was situated in an oil bath at a temperature of 130° C., during which the temperature of the solution rose to 125° C. The flow rate, which was adjusted with a high-pressure pump, was 700-800 ml/min under a pressure of 9 MPa. This hot solution was atomized and agglomerated through a single-fluid nozzle with a diameter of 0.7 mm under a pressure of 90 bar in an FSD spray dryer. An outlet air temperature of 96° C. was set up with an inlet air temperature of 145° C. A dry powder with excellent flow properties was obtained.

### Example 3

Solid solution of sulfathiazole in copolyvidone

10.0 kg of Kollidon VA 64 and 1.0 kg of mannitol were dissolved in 40.0 kg of demineralized water. 5.0 kg of finely ground sulfathiazole were suspended with vigorous stirring in this polymer solution. The brief heating took place by pumping the solution through a thin coiled pipeline with a diameter of 10 mm which was located in an oil bath at a temperature of 130° C., during which the temperature of the solution rose to 116° C. The flow rate, which was adjusted with a high-pressure pump, was 600-800 ml/min under a pressure of 9.1 MPa. This hot solution was atomized through a single-fluid nozzle with a diameter of 0.6 mm under a pressure of 90 bar in a spray dryer. An outlet air temperature of 55° C. was set up with an inlet air temperature of 115° C. A dry, free-flowing powder was obtained.

### Example 4

Solid solution of piroxicam in 1:1 copolyvidone/povidone

4.5 kg of Kollidon VA 64, 5.0 kg of Kollidon 30, 0.25 kg of nicotinamide and 0.5 kg of sodium lauryl sulfate were dissolved in 40.0 kg of demineralized water. 3.0 kg of finely ground piroxicam were suspended with vigorous stirring in this polymer solution. The brief heating took place by pumping the solution through a thin coiled pipeline with a diameter of 10 mm which was located in an oil bath at a temperature of 200° C., during which the temperature of the solution rose to 160° C. The flow rate, which was adjusted with a high-pressure pump, was 600-700 ml/min under a pressure of 9.1 MPa. This hot solution was atomized through a single-fluid nozzle with a diameter of 0.6 mm under a pressure of 90 bar in a spray dryer. An outlet air temperature of 70° C. was set up with an inlet air temperature of 125° C. A dry, free-flowing powder was obtained.

### Example 5

Solid solution of clotrimazole in povidone K 17

10.0 kg of Kollidon 17 PF, 0.3 kg of Lutrol F 68 (poloxamer 188) and 0.3 kg of sodium stearate were dissolved in 40.0 kg of demineralized water. 2.0 kg of finely ground clotrimazole were suspended with vigorous stirring in this polymer solution. The brief heating took place by pumping the solution through a thin coiled pipeline with a diameter of 10 mm which was located in an oil bath at a temperature of 135° C., during which the temperature of the solution rose to 115° C. This hot solution was atomized with a two-fluid nozzle in a spray fluidized bed dryer. An outlet air temperature of 65° C. was set up with an inlet air temperature of 100° C., A dry, relatively fine, very free-flowing powder was obtained.

### Example 6

Solid solution of cinnarizine in povidone

10.0 kg of Kollidon 30 and 0.5 kg of Cremophor RH 40 (product of the reaction of hydrogenated castor oil with 45 mol of ethylene oxide) were dissolved in 40.0 kg of demineralized water. 2.5 kg of cinnarizine were suspended with vigorous stirring in this polymer solution and then homogenized by treating with an Ultra-turrax for 15 min. A stream of this fine-particle suspension was heated to 60° C. in a heat exchanger and combined through a T-piece with a stream of water heated to 280° C. The ratio of the active ingredient-containing stream to the hot water stream was 1:2. The temperature of the hot solution upstream of the spray nozzle was 195° C. and the residence time at this temperature was 2.5 seconds. This hot solution was atomized through a single-fluid nozzle with a diameter of 0.6 mm under a pressure of 100 bar in a spray dryer. An outlet air temperature of 95° C. was set up with an inlet air temperature of 145° C. A dry, free-flowing powder was obtained.

### Example 7

Solid solution of ketoconazole in povidone/polyvinylcaprolactam

8.0 kg of Kollidon 30, 2.0 kg of polyvinylcaprolactam of K value 30 and 0.2 kg of polysorbate 80 and 0.1 kg of ascorbyl palmitate were dissolved in 40.0 kg of demineralized water. 2.0 kg of ketoconazole were suspended with vigorous stirring in this polymer solution and then homogenized by treating with an Ultra-turrax for 15 min. A stream of this fine-particle suspension was heated to 50° C. in a heat exchanger and combined with a stream of water heated to 240° C. in a mixing cell. The ratio of the active ingredient-containing stream to the hot water stream was 1:5. The temperature of the hot solution upstream of the spray nozzle was 200° C. and the residence time at this temperature was 2.0 seconds. This hot solution was atomized through a single-fluid nozzle with a diameter of 0.6 mm under a pressure of 100 bar in a spray dryer. An outlet air temperature of 95° C. was set up with an inlet air temperature of 145° C. A dry, free-lowing powder was obtained.

### Example 8

Solid solution of indometacin in povidone

10.0 kg of Kollidon 30, 0.2 kg of polyethylene glycol 6000 and 0.2 kg of Cremophor RH 40 were dissolved in 40.0 kg of demineralized water. 2.0 kg of indometacin were suspended with vigorous stirring in this polymer solution and then homogenized by treating with an Ultra-turrax for 15 min. A stream of this fine-particle suspension was heated to 50° C. in a heat exchanger and combined with a stream of water heated to 280° C. in a mixing cell. The ratio of the active ingredient-containing stream to the hot water stream was 1:1. The temperature of the hot solution upstream of the spray nozzle was 158° C. and the residence time at this temperature was 1.5 seconds. This hot solution was atomized through a single-fluid nozzle with a diameter of 0.6 mm under a pressure of 100 bar in a spray dryer. At the same time, Aerosil 200 was introduced as dust through a separate nozzle into the tower, with the ratio of solid from the solution to Aerosil being 99:1. An outlet air temperature of 82° C. was set up with an inlet air temperature of 143° C. A dry, free-flowing powder was obtained.

### Example 9

Solid solution of beta-carotene in povidone

8.0 kg of Kollidon 25, 1.5 kg of Cremophor RH 40, 0.5 kg of urea, 0.1 kg of ascorbyl palmitate and 0.05 kg of butylhydroxytoluene were dissolved in 30.0 kg of oxygen-free demineralized water. 1.0 kg of beta-carotene was suspended with vigorous stirring in this polymer solution and then homogenized by treating with an Ultra-turrax for 15 min. A stream of this fine-particle suspension was heated to 70° C. in a heat exchanger and combined with a stream of water heated to 230° C. in a mixing cell. The ratio of the active ingredient-containing stream to the hot water stream was 1:3. The temperature of the hot solution upstream of the spray nozzle was 190° C. and the residence time at this temperature was 3.0 seconds. This hot solution was atomized through a single-fluid nozzle with a diameter of 0.7 mm under a pressure of 100 bar in a spray dryer. An outlet air temperature of 69° C. was set up with an inlet air temperature of 120° C. A dry, free-flowing powder was obtained.

### Example 10

Solid solution of theophylline in urea

10.0 kg of urea were dissolved in 40.0 kg of demineralized water. 3.0 kg of finely ground theophylline were suspended with vigorous stirring in this polymer solution. The brief heating took place by pumping the solution through a thin coiled pipeline with a diameter of 3 mm which was situated in an oil bath at a temperature of 155° C., during which the temperature of the solution rose to 150° C. The flow rate, which was set with a high-pressure pump, was 500-600 ml/min. This hot solution was atomized through a single-fluid nozzle with a diameter of 0.5 mm under a pressure of 100 bar in a spray dryer. An outlet air temperature of 77° C. was set up with an inlet air temperature of 125° C. A dry, free-flowing powder was obtained.

### Comparative Example 1

Solid dispersion of theophylline in povidone

10.0 kg of Kollidon 30 were dissolved in 40.0 kg of demineralized water. 5.0 kg of theophylline were suspended with vigorous stirring in this polymer solution. This suspension was atomized through a single-fluid nozzle with a diameter of 0.5 mm under a pressure of 100 bar in a spray dryer. An outlet air temperature of 90° C. was set up with an inlet air temperature of 160° C. A dry, free-flowing powder was obtained.

### Comparative Example 2

Solid dispersion of carbamazepine in povidone

10.0 kg of Kollidon 30 were dissolved in 40.0 kg of demineralized water. 5.0 kg of carbamazepine were suspended with vigorous stirring in this polymer solution. This suspension was atomized and agglomerated through a two-fluid nozzle in an FSD spray dryer. An outlet air temperature of 88° C. was set up with an inlet air temperature of 155° C. A dry powder was obtained.

TABLE 1

| | Microscopic | DSC | X-ray |
|---|---|---|---|
| Examples | assessment | investigation | diffractometry |
| Example 1 | no crystals | No active | amorphous |
| Theophylline | | ingredient peak | |
| | | at 270-274° C. | |
| Example 2 | no crystals | No active | amorphous |
| Carbamazepine | | ingredient peak | |
| | | at 191° C. | |
| Example 3 | no crystals | No active | amorphous |
| Sulfathiazole | | ingredient peak | |
| | | at 202° C. | |
| Example 4 | no crystals | No active | amorphous |
| Piroxicam | | ingredient peak | |
| | | at 205° C. | |
| Example 5 | no crystals | No active | amorphous |
| Clotrimazole | | ingredient peak | |
| | | at 148° C. | |
| Example 6 | no crystals | No active | amorphous |
| Cinnarizine | | ingredient peak | |
| Example 7 | no crystals | No active | amorphous |
| Ketoconazole | | ingredient peak | |
| | | at 146° C. | |
| Example 8 | no crystals | No active | amorphous |
| Indometacin | | ingredient peak | |
| | | at 155° C. | |
| Example 9 | no crystals | No active | amorphous |
| Beta-carotene | | ingredient peak | |
| Example 10 | no crystals | No active | amorphous |
| Theophylline | | ingredient peak | |
| | | at 270-274° C. | |
| Comparative | crystals | Active ingredient | crystalline |
| example 1 | | peak at | fractions |
| | | 270-274° C. | |
| Comparative | crystals | Active ingredient | crystalline |
| example 2 | | peak at 191° C. | fractions |

### Example 11

Theophylline tablets

2.1 kg of theophylline solid solution from Example 1 were mixed with 1.5 kg of Ludipress® LCE (coprocessed product of 93% lactose, 3.5% povidone, 3.5% crospovidone), 0.03 kg of colloidal silica (Aerosil 200, from Degussa), 0.15 kg of crospovidone (Kollidon CL, from BASF) and 0.03 kg of magnesium stearate in a Turbula mixer for 10 min and compressed to tablets under a compressive force of 18 kN using a Korsch PH 106 type rotary tablet press. The tablets had a diameter of 10 mm and a weight of 331 mg.

Tablets were also compressed analogously using the powder from Comparative example 1 and with pure theophylline crystals.

The resistance to crushing and the active ingredient release in a USP 2004 paddle apparatus were determined for the tablets.

| | Resistance to | Release after | Release after |
|---|---|---|---|
| Product | crushing | 15 min in % | 30 min in % |
| Example 1 | 225 | 62 | 99 |
| Comparative example 1 | 174 | 42 | 80 |
| Theophylline crystals | 140 | 34 | 78 |

### Example 14

Carbamazepine capsules

180 g of carbamazepine solid solution from Example 2 were mixed with 100 g of calcium hydrogen phosphate, 1.5 g of Aerosil 200 and 20 g of Kollidon CL in a Turbula mixer for 10 min and packed into gelatin capsules in an amount of 301.5 mg.

Capsules were also produced analogously with the powder from Comparative example 2 and with pure carbamazepine crystals.

The active ingredient release in a USP 2004 paddle apparatus was determined for the capsules.

| | Release after | Release after |
|---|---|---|
| Product | 15 min in % | 30 min in % |
| Example 2 | 55 | 101 |
| Comparative example 2 | 35 | 66 |
| Carbamazepine crystals | 22 | 53 |

1-28. (canceled)

29. A process for producing solid solutions in powder or granule form of slightly soluble substances, wherein said slightly soluble substance is in the form of a molecular dispersion in an excipient matrix, comprising atomizing a solution of the active ingredient and matrix excipients by heating an aqueous suspension of said slightly soluble substance in the presence of the matrix excipients to temperatures above the boiling point under atmospheric pressure, dissolving the slightly soluble substance, and converting said solution of slightly soluble substance and matrix excipients by atomizing and drying into a solid form, wherein the temperature of the spray solution before feeding into the atomizing apparatus is in the range of from 90° C. to 350° C.

30. The process of claim 29, wherein the temperature of the spray solution before atomizing is in the range of from 110 to 300° C. and said active ingredient is in dissolved form.

31. The process of claim 29, wherein the temperature of the spray solution before atomizing is in the range of from 120 to 250° C. and said active ingredient is in dissolved form.

32. The process of claim 29, wherein the residence time of said active ingredient at temperatures above 90° C. is less than 180 seconds.

33. The process of claim 29, wherein the residence time of said active ingredient at temperatures above 90° C. is less than 60 seconds.

34. The process of claim 29, wherein the residence time of said active ingredient at temperatures above 90° C. is less than 15 seconds.

35. The process of claim 29, wherein the concentration of said slightly soluble substance in the excipient matrix is from 1 to 50% by weight

36. The process of claim 29, wherein the concentration of said slightly soluble substance in the excipient matrix is from 10 to 50% by weight.

37. The process of claim 29, wherein the concentration of said slightly soluble substance in the excipient matrix is from 20 to 50% by weight.

38. The process of claim 29, wherein said matrix excipients comprise amide groups.

39. The process of claim 29, wherein said matrix excipients comprise homo- or copolymers of N-vinylpyrrolidone, N-vinylcaprolactam, N-vinylformamide, or N-vinylacetamide.

40. The process of claim 29, wherein said solid solutions further comprise surfactants having a HLB above 10.

41. The process of claim 29, wherein said solid solutions further comprise cosolubilizers having a HLB below 10.

42. The process of claim 29, wherein said spray solution comprises an adsorbent.

43. The process of claim 29, wherein said suspension of the active ingredient is heated by a heat exchanger.

44. The process of claim 29, wherein heating of said suspension of the active ingredient is achieved by mixing with a hot stream of liquid or a hot stream of vapor.

45. The process of claim 44, wherein the ratio of said suspension of the active ingredient to said hot stream of liquid is from 9:1 to 1:9.

46. The process of claim 44, wherein the ratio of said suspension of the active ingredient to said hot stream of liquid is from 7:3 to 3:7.

47. The process of claim 44, wherein the temperature of said hot stream of liquid or vapor is in the range of from 110 to 500° C.

48. The process of claim 44, wherein the temperature of said hot stream of liquid or vapor is in the range of from 150 to 400° C.

49. The process of claim 48, wherein the temperature of said hot stream of liquid or vapor is in the range of from 180 to 300° C.

50. The process of claim 29, wherein said drying is achieved by atomizing drying or of by fluidized bed spray granulation.

51. The process of claim 29, wherein an adsorbent or dusting agent is blown into the spray tower during atomizing drying.

52. The process of claim 29, wherein said slightly soluble active ingredients are medicinal substances, vitamins, carotenoids, or nutraceuticals.

53. A solid solution in the form of a powder or granules prepared by the process of claim 29.

54. The powder or granules of claim 53, comprising a) 1 to 50% by weight of active ingredient, b) 10 to 99% by weight of a water-soluble matrix excipient, c) 0 to 30% by weight of solubilizers, d) 0 to 30% by weight of cosolubilizers and e) 0 to 50% by weight of further customary excipients.

55. A pharmaceutical dosage form, food product, or dietary supplement comprising the powder or granules of claim 53.
